(19) United States
(12) Patent Application Publication
(10) Pub. No.: US 2013/0216593 A1
(43) Pub. Date: Aug. 22, 2013

(54) SYSTEMS, METHODS, AND DEVICES FOR PLASMID GENE TRANSFECTION USING POLYMER-MODIFIED MICROBUBBLES

(71) Applicants: Mark Andrew Borden, Boulder, CO (US); Shashank Ramesh Sirsi, New York, NY (US)

(72) Inventors: Mark Andrew Borden, Boulder, CO (US); Shashank Ramesh Sirsi, New York, NY (US)

(73) Assignee: The Trustees of Columbia University in the City of New York, New York, NY (US)

(21) Appl. No.: 13/818,749

(22) PCT Filed: Aug. 26, 2011
(86) PCT No.: PCT/US11/49455

§ 371 Date: Apr. 26, 2013

Related U.S. Application Data
(60) Provisional application No. 61/377,941, filed on Aug. 28, 2010.

(51) Int. Cl. A61K 9/107; A61K 31/7088

(52) U.S. Cl. CPC A61K 9/1075; A61K 31/7088
USPC 424/400; 514/44 R

(57) ABSTRACT

Thiolated polyethylenimine (PEI) polymers can be covalently attached to lipid shell microbubbles. The PEI polymer can be modified with polyethylene glycol (PEG) chains to improve biocompatibility. The covalent attachment of the PEI polymer to the microbubble shell can result from a bond between a free sulfhydryl group (SH) of the thiolated PEI and a free maleimide group on the microbubble shell. DNA can be electrostatically bound to the PEI polymers to form polyplexes. A plurality of the polyplex-microbubble hybrids can be injected into a patient and can be imaged via ultrasound. While circulating in the bloodstream, and in particular, within a region of interest, high-pressure, low-frequency acoustic energy can be applied, thereby causing destruction by cavitation. Such cavitation can transiently increase the permeability of the endothelial vasculature thereby allowing plasmid DNA of the polyplexes carried by the microbubbles to be delivered to targeted cells.

### CROSS-REFERENCE TO RELATED APPLICATIONS

The present application claims the benefit of U.S. Provisional Application No. 61/377,941, filed Aug. 28, 2010, which is hereby incorporated by reference herein in its entirety.

### FIELD

The present disclosure relates generally to genetic modification of targeted cells via DNA delivery thereto, and, more particularly, to plasmid gene transfection using polymer-modified microbubbles.

### BACKGROUND

Microbubbles are gas-filled spheres, typically 1-10 μm in diameter, which circulate in the bloodstream when injected systemically. When insonified at ultrasonic frequencies, microbubbles may undergo cavitation or volumetric oscillations. Stable cavitation is marked by microbubble persistence over the acoustic pulse train and generally results in relatively mild viscous effects, such as microstreaming. Inertial cavitation may occur at higher acoustic powers and involves rapid microbubble collapse and fragmentation to produce shock waves, water jets and other intense, highly localized effects. Both forms of microbubble cavitation can create pores in the endothelial layer (sonoporation) to aid in drug and gene delivery.

Sonoporation can be an effective method of promoting extravasation of large macromolecules, such as plasmid DNA, to improve delivery to tissue beyond the vasculature. The tradeoff comes as stable cavitation creates less “collateral damage” to nearby tissue, while inertial cavitation provides a greater extent of extravascular delivery. Sonoporation may be suited for site-specific drug delivery, since permeabilization of vasculature and delivery of cargo only occurs at sites where ultrasound is applied and microbubbles are present. By spatially and temporally controlling the application of ultrasound energy, gene uptake can be targeted to specific regions. Since microbubble cavitation also results in an acoustic emission, sonoporation can be guided and tracked by ultrasound imaging. Image-guided sonoporation may be particularly useful for tumor-targeted drug and gene therapy. However, systemic gene delivery has been largely inefficient due to rapid clearance of nucleic acids from the bloodstream via the mononuclear phagocyte system (MPS) and enzymatic degradation.

### SUMMARY

Systems, methods, and devices for plasmid gene transfection using polymer-modified microbubbles are disclosed herein. Thiolated polyethyleneimine (PEI) polymers can be covalently attached to lipid shell microbubbles. The PEI polymer can be modified with polyethylene glycol (PEG) chains to improve biocompatibility. The covalent attachment of the PEI polymer to the microbubble shell can result from a bond between a free sulfhydryl group (SH) of the thiolated PEI and a free maleimide group on the microbubble shell. DNA can be electrostatically bound to the PEI polymers to form polyplexes. In addition, the microbubbles can be size-selected to have diameters of 4-5 μm or 6-8 μm for improved circulation persistence, echogenicity, and sonoporation capability.

A plurality of the polyplex-microbubble hybrids can be injected into a patient and can be imaged via ultrasound. While circulating in the bloodstream, and in particular, within a region of interest, high-pressure, low-frequency acoustic energy can be applied, thereby causing destruction by cavitation. Such cavitation can transiently increase the permeability of the endothelial vasculature thereby allowing DNA plasmids of the polyplexes carried by the microbubbles to be delivered to targeted cells. This technique may find particular application for targeted plasmid DNA delivery to cancerous tumors.

In embodiments, a microbubble for gene transfection can include a gas-filled core region, a shell, and one or more polyplex structures. The shell can surround the gas-filled core region and can comprise a lipid formulation. The one or more polyplex structures can be covalently attached to the shell. A plurality of these microbubbles can be used as part of a gene transfection suspension.

In embodiments, a system for gene transfection can include a plurality of microbubbles and an ultrasound imaging system. Each microbubble can have a gas-filled core region, a shell, and one or more polyplex structures. The shell can surround the gas-filled core region and can include a lipid formulation. The one or more polyplex structures can be covalently attached to the shell. The ultrasound imaging system can be configured to image vasculature and the plurality of microbubbles therein during a first mode of operation. The ultrasound imaging system can also be configured to apply a high-pressure, low-frequency ultrasound pulse during a second mode of operation such that the microbubbles in the vasculature are destroyed.

In embodiments, a method for forming microbubbles for gene transfection can include emulsifying a lipid formulation with a gas so as to produce a plurality of microbubble shells, each shell surrounding a respective gas-filled core region. The method can further include covalently attaching one or more polymers to each of the shells. The method can also include electrostatically binding DNA to the one or more polymers so as to form one or more polyplex structures.

In embodiments, a method of gene transfection can include injecting a plurality of microbubbles into a patient, and applying a high-pressure, low-frequency ultrasound pulse to a region of interest in the patient so as to destroy microbubbles in said region of interest. Each microbubble can have a gas-filled core region surrounded by a shell. The shell can be comprised of a lipid formulation and can have one or more polyplex structures covalently attached thereto.

Objects and advantages of embodiments of the disclosed subject matter will become apparent from the following description when considered in conjunction with the accompanying drawings.

### BRIEF DESCRIPTION OF DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements.

FIG. 1 is a simplified diagram showing aspects of a polyplex-microbubble hybrid, according to one or more embodiments of the disclosed subject matter.

FIG. 2 is a flow diagram of a process for forming microbubbles, according to one or more embodiments of the disclosed subject matter.

FIGS. 3A-3B shows number- and volume-weighted distributions, respectively, of a size-selected microbubble suspension following conjugation of PEG-PEI-SH, according to one or more embodiments of the disclosed subject matter.

FIGS. 4A-4B show bright field and fluorescence images, respectively, of microbubbles loaded with F-PEG-PEI-SH, according to one or more embodiments of the disclosed subject matter.

FIG. 5 is a density scatter plot from forward and side scattering during flow cytometric analysis of fluorescent PEG-PEI-SH binding to maleimide containing microbubbles, according to one or more embodiments of the disclosed subject matter.

FIGS. 6A-6C are graphs of median fluorescent intensity (MFI) versus time of the microbubbles from the gated regions B, C, and D, respectively, according to one or more embodiments of the disclosed subject matter.

FIG. 7 is a graph of zeta-potential for microbubbles without PEG-PEI-SH loading, with PEG-PEI-SH loading, and with PEG-PEI-SH and DNA loading for different maleimide concentrations, according to one or more embodiments of the disclosed subject matter.

FIG. 8 is a graph of DNA loading capacity of microbubbles with PEG-PEI-SH loading for different maleimide concentrations, according to one or more embodiments of the disclosed subject matter.

FIGS. 9A-9B are time intensity curves for control microbubbles and targeted microbubbles, respectively, in a region of interest in the time surrounding the application of a destruction pulse, according to one or more embodiments of the disclosed subject matter.

FIGS. 10A-10B are ultrasound images of a tumor in the region of interest for control microbubbles and targeted microbubbles, respectively, according to one or more embodiments of the disclosed subject matter.

FIGS. 11A-11B are simplified schematic diagrams of polyplex-loaded microbubbles within a patient vasculature before and after application of high-intensity, low-frequency ultrasound, according to one or more embodiments of the disclosed subject matter.

FIG. 11C is a simplified schematic diagram of plasmid DNA transfection mechanisms into a cell after application of high-intensity, low-frequency ultrasound, respectively, according to one or more embodiments of the disclosed subject matter.

FIGS. 12A-12B are fluorescence images illustrating transfection of DNA in a cell plate outside of an ultrasound focus and within the ultrasound focus, according to one or more embodiments of the disclosed subject matter.

FIG. 12C is a graph of the fluorescence intensities measured in FIGS. 12A-12B.

FIG. 13 is a simplified schematic diagram of a system for gene transfection using polyplex-loaded microbubbles, according to one or more embodiments of the disclosed subject matter.

FIG. 14 is a flow diagram of a process for gene transfection using polyplex-loaded microbubbles, according to one or more embodiments of the disclosed subject matter.

FIG. 15 is an image of a mouse tumor transfected with a bioluminescent reporter gene, according to one or more embodiments of the disclosed subject matter.

FIG. 16 is an ultrasound image of a mouse kidney with different regions of interest indicated therein, according to one or more embodiments of the disclosed subject matter.

FIG. 17 shows ultrasound B-mode images (column 1), contrast images (column 2), and B-mode/contrast overlays (column 3) for control microbubbles (row A), PEI-microbubbles without DNA (row B), and polyplex-loaded microbubbles (row C) injected into a mouse kidney, according to one or more embodiments of the disclosed subject matter.

FIGS. 18A-18B are time-intensity curves for PEI-microbubbles and polyplex-microbubbles, respectively, for different maleimide concentrations, according to one or more embodiments of the disclosed subject matter.

FIG. 19 is a graph of maximum signal intensity for PEI-microbubbles and polyplex-microbubbles at different maleimide concentrations, according to one or more embodiments of the disclosed subject matter.

FIG. 20 is a graph of half-life for PEI-microbubbles and polyplex-microbubbles at different maleimide concentrations, according to one or more embodiments of the disclosed subject matter.

FIG. 21A shows time-intensity and time-fluctuation curves for control microbubbles, according to one or more embodiments of the disclosed subject matter.

FIGS. 21B-C show time-intensity and time-fluctuation curves for PEI-microbubbles and polyplex-microbubbles, respectively, having 0.5% maleimide, according to one or more embodiments of the disclosed subject matter.

FIGS. 21D-E show time-intensity and time-fluctuation curves for PEI-microbubbles and polyplex-microbubbles, respectively, having 2% maleimide, according to one or more embodiments of the disclosed subject matter.

FIGS. 21F-G show time-intensity and time-fluctuation curves for PEI-microbubbles and polyplex-microbubbles, respectively, having 5% maleimide, according to one or more embodiments of the disclosed subject matter.

FIG. 22 is a graph of Dₒ determined from the time-intensity and time-fluctuation curves for control microbubbles, PEI-microbubbles, and polyplex-microbubbles, according to one or more embodiments of the disclosed subject matter.

FIG. 23 is a graph of adhesion ratio calculated from k₂ and k₃ values determined from the time-intensity and time-fluctuation curves for control microbubbles, PEI-microbubbles, and polyplex-microbubbles, according to one or more embodiments of the disclosed subject matter.

FIGS. 24A-24D are images of luciferase expression in mice after transfection with 5% maleimide polyplex-microbubbles and ultrasound, 5% maleimide polyplex-microbubbles without ultrasound, plasmid DNA only with ultrasound, and after no treatment (control), respectively, according to one or more embodiments of the disclosed subject matter.

FIG. 25 is a graph of relative luciferase expression for the transfection conditions applied to the mice in the images of FIGS. 24A-24D.

FIG. 26 is a graph of ex vivo quantification of luciferase expression for the transfection conditions applied to the mice in the images of FIGS. 24A-24D.

FIG. 27 is a simplified schematic diagram showing layer-by-layer assembly for microbubble formation, according to one or more embodiments of the disclosed subject matter.

FIG. 28 is a graph of zeta potential as function of the number of deposition steps in the layer-by-layer assembly of FIG. 27, according to one or more embodiments of the disclosed subject matter.

FIG. 29 is a graph of DNA loading enhancement as a function of the number of layers in the layer-by-layer assembly of FIG. 27, according to one or more embodiments of the disclosed subject matter.

FIG. 30 shows fluorescence microscopy images of microbubbles produced using the layer-by-layer assembly of FIG. 27, according to one or more embodiments of the disclosed subject matter.

### DETAILED DESCRIPTION

Microbubble-based ultrasound contrast agents can serve as gene and/or drug carriers for targeted delivery applications and for non-viral gene delivery by improving the efficiency of plasmid DNA transfection in cells. The use of plasmid DNA for therapeutic and clinical applications has been hindered by low transfection efficiencies. The disclosed polymer modified microbubbles can have significantly increased payloads to deliver plasmid DNA to targeted tissue and can improve transfection of plasmid DNA via sonoporation. The polymer-modified microbubbles can also promote intracellular trafficking of plasmid DNA to nuclei of target cells, presumably increasing the levels of plasmid gene expression in a target specific manner.

High molecular weight (e.g., 25 kDa) polyethyleneimine (PEI) can be thiolated and mixed with anionic plasmid DNA to form polyplex structures. The polyplex structures can be covalently attached to the microbubble surface by maleimide chemistry to form polyplex-microbubble hybrids. Additionally or alternatively, low molecular weight PEI (e.g., <2 kDa) can be thiolated and form larger aggregate structures (e.g., >25 kDa) stably linked through disulfide bonds between free thiol groups. These aggregate structures with DNA bound thereto could also be covalently attached to microbubbles by maleimide surface chemistry to from polyplex-microbubble hybrids. The larger aggregate structures could bind more DNA and enhance transfection efficiency. In addition, the bonds are enzymatically cleavable, which may facilitate degradation of the larger aggregate structures into smaller and less toxic PEI monomer units after delivery of their DNA payload.

The polyplex-microbubble hybrids can be injected into the patient and allowed to circulate in the patient's bloodstream. Ultrasound can be applied over a region of interest (e.g., an area including a tumor or other desired area for DNA transfection) at a time after the injection for imaging the region of interest. The polyplex-loaded microbubbles can also be used as a contrast agent thereby allowing imaging within the bloodstream in order to determine the persistence of the microbubbles in the bloodstream. While circulating in the blood stream, acoustic energy can cause microbubble destruction by cavitation that transiently increases the permeability of the endothelial vasculature, allowing macromolecules such as plasmids to be delivered to target cells. Transfection of the DNA may be localized to those regions of interest exposed to the acoustic energy. Such a technique can find particular application for targeted plasmid DNA delivery to cancerous tumors, for example.

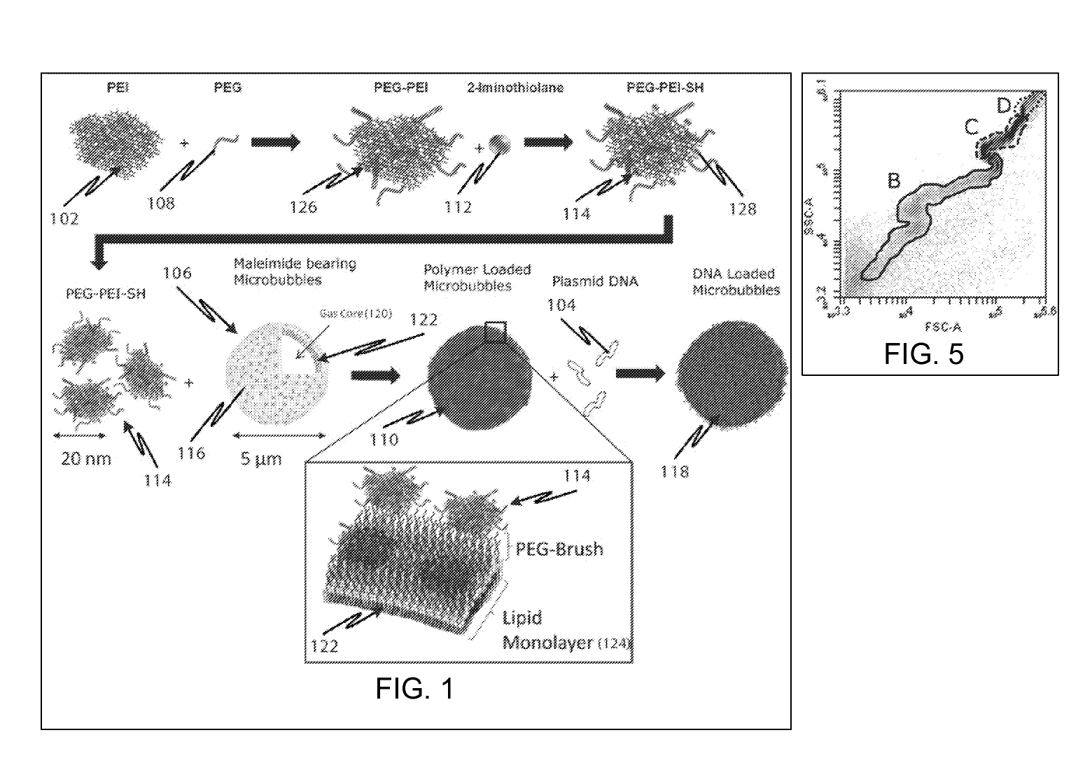

Referring to FIG. 1, PEI 102, which is a highly cationic branched polymer, can electrostatically bind plasmid DNA 104 thereto so as to form a compact structure (i.e., polyplex) that can be attached to the shell of the microbubble 106. The binding with PEI can protect the DNA from enzymatic degradation and provide for easier internalization of the plasmid 104 into the cell. In addition, the use of PEI can promote endocytosis, endosomal escape of DNA into the cell cytoplasm by the “proton sponge” effect, and localization within the nucleus. Furthermore, PEI promotes intracellular trafficking of plasmid DNA to the nucleus of cells where they are able to function.

Due to the high cationic charge of the polymer backbone, PEI-based vectors are rapidly cleared from circulation and are potentially cytotoxic in high doses. The biocompatibility can be dramatically improved by the addition of inert polyethylene glycol (PEG) chains 108 so as to ameliorate the surface charge and reduce complement activation, thereby improving biocompatibility. Pegylation of PEI can improve solubility of the polyplexes, sterically inhibit opsonization of serum proteins, and generally improve the circulation time and transfection efficiency of polyplexes in vivo. Other methods of reducing toxicity can also be employed, such as, but not limited to cross linking low-molecular-weight PEI molecules to make biodegradable PEI-based vectors. For example, low molecular weight PEI can be formed into an aggregate structure using cross-linking by biodegradable bonds (e.g., disulfide bonds) to reduce PEI toxicity in vivo.

PEI polymers 102 can be covalently coupled to the lipid-coated microbubbles to create PEI-microbubble hybrids 110. The PEI 102 can be thiolated (i.e., to have a free sulfhydryl group (—SH) 128) using 2-iminothiolane 112 for covalent binding to PEG-tethered maleimide (Mal) groups 116 on the shell 122 of the microbubble 106. The microbubbles can be size-selected to improve their circulation persistence, echogenicity, and sonoporation capability. For example, the microbubbles can be selected such that most (or substantially all) of the microbubbles in a suspension have diameters falling within one of the ranges of approximately 4-5 μm and 6-8 μm.

The plasmid DNA 104 can be loaded onto the PEI polymer 102 to form polyplexes before or after attachment of the PEI polymer 102 to the shell 122 of the microbubble 106 so as to form a polyplex-microbubble hybrid 118. The disclosed microbubbles can carry more DNA than unmodified microbubbles and can have higher transfection efficiencies for the plasmid DNA. Unmodified microbubble vehicles may have a finite surface area and therefore limited loading capacity, since nucleic acids are not soluble in the gas phase and therefore cannot be encapsulated within the microbubble core. For example, loading capacity of unmodified lipid-coated microbubbles is approximately 80 μm² for a 5 μm diameter microbubble. Considering a “hit-and-stick” adsorption model, the surface density is approximately 0.0001 pg/μm² for a 10 kbp DNA plasmid, resulting in an estimated maximum loading density of approximately 0.01 pg/microbubble.

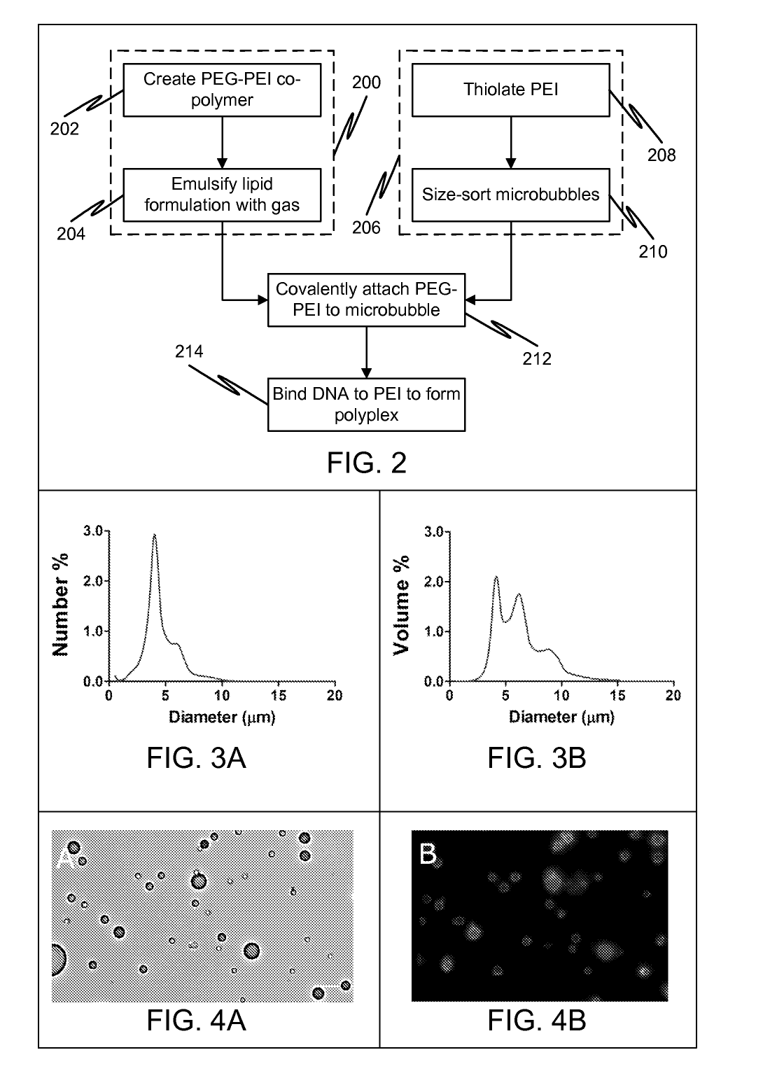

Referring to FIG. 2, a process for forming a DNA-loaded microbubble is illustrated. The process begins at 202 where the PEI polymer 102 is pegylated. PEG chains 108 can be added to the PEI 102 using amine-reactive polyethylene glycol succinimidyl ester (NHS-PEG) at a 10:1 molar ratio to PEI to create the PEG-PEI co-polymer 126. For example, cationic branched polymer PEI with a molecular weight (MW) of 25 kDa and NHS-PEG with a MW of 5 kDa can be used. The PEI polymer can be dissolved in phosphate buffered saline (PBS), with the pH thereof adjusted to 8.4, to a concentration of, for example, 10 mg/mL. 100 mg of NHS-PEG can be dissolved in 300 μL of dimethylformamide (DMF). The NHS-PEG solution can then be added to the PEI solution drop-wise while rigorously mixing for a period of time, such as, 1 hour. NHS esters on the PEG chains are reactive compounds that form stable amide bonds with amine groups on the PEI structure, thus creating PEG-PEI copolymers when mixed. The resulting solution can be dialyzed using dialysis tubing with a molecular weight cutoff (MWCO) of 14-16 kDa. The dialyzed solution can be subsequently frozen and lyophilized prior to thiolation.

The 25-kDa, branched PEI can have an amine-to-phosphate ratio (N/P) of 5 to 6, although other N/P ratios are also possible according to one or more contemplated embodiments (e.g., N/P ratios of 0.1 to 50). This may efficiently encapsulate DNA to form nanoparticles with diameters <200 nm, suitable for clathrin-mediated cellular uptake. For example, 25 k-kDa PEI with 5.8-kbp plasmid DNA (N/P=6) can result in roughly 3.5 plasmids and 30 PEI molecules per 70±10 nm diameter polyplex. This corresponds to roughly 2.0×10⁻⁵ pg DNA per polyplex. In another example, low molecular weight PEI (e.g., <2 kDa) polymers are thiolated and formed into larger aggregate structures (e.g., >25 kDa) stably linked by disulfide bridges formed between free thiol groups on the SH-PEG-PEI complex. Other transfection polymers besides the above described PEI can also be used according to one or more contemplated embodiments.

The process can then proceed to 204, where the PEG-PEI polymers 126 can be modified with 2-iminothiolane 112 (i.e., Trauts reagent), which can introduce free SH groups 128 in a thiolation process. The introduced SH groups 128 on the PEG-PEI polymer 126 allow for binding to the maleimide-expressing shell 122 of microbubble 106, in order to chemically link the polymers to the microbubbles 106. The Trauts reagent 112 can be reacted with the PEG-PEI polymers 126 at, for example, a 50:1 molar excess. For example, PEG-PEI can be dissolved at a concentration of 10 mg/mL in PBS buffer (pH 6.5) containing 5 mM ethylenediaminetetraacetic acid (EDTA). 2-iminothiolane (i.e., Trauts reagent) can be dissolved in PBS buffer to 1 mg/mL and added drop-wise to the PEG-PEI solution at a 50:1 molar ratio while rigorously mixing. The solution can be mixed for 1 hour and dialyzed for 48 hours using dialysis tubing with a 4-6 kDa MWCO. The resulting solution can be subsequently freeze-dried to obtain the final thiolated PEG-PEI polymers (i.e., PEG-PEI-SH 114).

The process also includes forming the microbubbles at 206, which may occur before, concurrently with, or after the formation of the thiolated polymers 114 at 200. The formation of the microbubbles can begin at 208 where a lipid formulation is emulsified with a gas. For example, the lipid formulation can be emulsified with a hydrophobic gas, such as SF₆ or perfluorobutane (PFB). The lipid formulation can include, for example, lipid molar ratios of 90% 1,2-distearoyl-sn-glycero-3-phosphocholine (DSPC) and 10% 1,2-distearoyl-sn-glycero-3-phosphoethanolamine-N-[maleimide(polyethylene glycol)-2000] (DSPE-PEG2K-Mal). In another example, the lipid formulation can include 90% DSPC, between 0.5% and 5% DSPE-PEG2K-Mal, and the remainder (i.e., 5% to 9.5%) 1,2-distearoyl-sn-glycero-3-phosphoethanolamine-N-[amino(polyethylene glycol)-2000] (DSPC-PEG2K). The maleimide group 116 is a reactive species that binds to SH groups 128, thereby enabling covalent coupling of PEG-PEI-SH polymers 114 to the microbubble shell 122. The composition of the lipid formulation can be altered with the percentage of DSPE-PEG2K-Mal varying between 0.5% and 5%, in which case the amount of DSPE-PEG2K can be increased so that the DSPE-PEG based lipids constitutes approximately 10 mol % of the overall lipid composition.

The constituent solutions for the various lipid components can be dissolved and mixed at the appropriate ratios in chloroform in a sealed 3-mL glass serum vial to 1 mg total lipid per vial. The resulting lipids can be dried and re-suspended in 2 mL of 0.01 M PBS buffer containing 10 vol % glycerol and 10 vol % propanediol. The lipid solution can then be warmed to approximately 60° C. and briefly sonicated to disperse the lipid in a bath sonicator. The air headspace can be exchanged with a hydrophobic gas, such as PFB, using a gas exchange apparatus. The pressure in the vial can be vented briefly to the atmosphere to relieve pressure. The microbubbles 106 can then be formed by shaking in a vial mixer or sonicating using a sonicator. Microbubble solutions from individual vials can be combined together for further processing, for example, in a 12-mL or 30-mL syringe.

The process can proceed to 210 where the generated microbubble suspension can be size-sorted to select microbubbles having diameters within a desired range. The produced microbubbles can be size sorted using a process of differential centrifugation or other microbubble size separation techniques. For example, a population of microbubbles having diameters predominantly in the range between 4 and 10 μm (e.g., a mean diameter of 4.5 um) can be selected. Microbubbles with diameters of 4-5 μm and 6-8 μm may enjoy superior circulation persistence in the bloodstream of a patient. For example, lipid vesicles and microbubbles less than 4 μm in diameter can be removed using a centrifugation method. For example, the microbubble suspension can be repeatedly centrifuged at 90 relative centrifugal force (RCF) for 1 minute using a bucket-rotor centrifuge. After every repetition, the microbubble cake can be saved and infranatant discarded. The final microbubble suspension can be diluted in PBS buffer (pH 6.5) containing 1 mM EDTA.

In another example, microbubbles greater than 10-μm diameter can be removed by performing a centrifugation cycle at 30 RCF for 1 minute. The infranatant consisting of less than 10-μm diameter microbubbles can be saved and re-dispersed in 30 mL PBS, while the cake can be discarded. Next, microbubbles of greater than 6-μm diameter can be removed by performing a centrifugation cycle at 70 RCF for 1 minute. The infranatant consisting of less than 6-μm diameter microbubbles can be saved and re-dispersed to 30 mL PBS. The cake can be discarded. Microbubbles of less than 4-μm diameter can be removed by centrifuging at 160 RCF for 1 minute. This may be repeated, for example, about 5-10 times, or until the infranatant is no longer turbid, thus indicating that most of the microbubbles having a diameter less than 4 μm have been removed. After each cycle, the infranatant can be discarded, and the cake can be re-dispersed in filtered PBS. The final cake can be concentrated to a 1-mL volume of 20 vol % glycerol solution in PBS and stored in a 2-mL scintillation vial with headspace having the same gas as the microbubble core (e.g., PFB).

After formation of the thiolated polymers 114 and the microbubbles 106, the process can proceed to 212, where the polymers 114 are covalently bound to the microbubble shell 122. The microbubbles 106 can, in effect, be coated with PEG-PEI-SH polymers 114 by covalently coupling the maleimide end-groups 116 on the microbubble surface 122 to the thiol groups 128 of the PEG-PEI-SH polymers 114. For example, the polymers 114 can be dissolved to 10 mg/mL in PBS buffer (pH 6.5) containing 1 mM EDTA. Maleimide-bearing microbubbles 106 can be added drop-wise to the polymer solution while gently mixing. The resulting suspension can be gently mixed for an additional time period, for example, 24 hours. A molar excess of 10:1 PEI:maleimide ratio can be used to prevent aggregation of the microbubbles. Microscopy, for example, fluorescence microscopy, can be used to confirm deposition of the PEG-PEI-SH 114 polymer onto the microbubble shell 122.

At 214, DNA 104 may be bound to the PEI 102 to form polyplexes. Although the binding of DNA 104 to the PEI is shown in FIGS. 1-2 as occurring after the PEI polymer 102 is attached to the microbubble 106, it is also possible to bind the DNA 104 to the PEI before attaching the PEI polymer 102 to the microbubble 106. Thus, the DNA 104 may be bound to the PEI after 204 and before 212, so as to form a polyplex which is then covalently bonded to the microbubble shell 122. DNA can be rinsed by ethanol extraction and re-suspended in PBS. Branched PEI can be added dropwise while vortexing to form the polyplexes. Polyplexes can be isolated from free PEI by centrifugation, chromatography, and/or dialysis. Approximately 100-nm diameter particles can be loaded onto microbubbles (e.g., through avidin-biotin linkage) at approximately 10,000 nanoparticles per microbubble. This result corresponds well to the available surface area of a microbubble. Polyplex loading can lead to approximately 0.2 pg-DNA/microbubble, which is 20-fold greater than that achieved for naked DNA.

The resulting DNA-loaded microbubbles 118 can have increased loading capacities, for example, up to four times as much DNA per unit area (i.e., μm²) as cationic microbubbles made with 1,2-stearoyl-3-trimethylammoniumpropane (DSTAP) lipids. The polymer-modified microbubbles remain echogenic and show equal circulation persistence times as compared to unmodified microbubbles when the surface is loaded with DNA. Such microbubbles can be useful in a number of therapeutic, diagnostic, and industrial applications, including, but not limited to target specific gene delivery applications for research purposes and the delivery of therapeutic plasmids for clinical applications.

Microbubble size distributions and concentrations were determined by laser light obscuration and scattering. 2-μL samples of each microbubble suspension were diluted into a 30-mL flask under mild mixing. The total amount of maleimide in the final sample was estimated from the total surface area calculated by the sizing measurement, the initial DSPE-PEG2k-Mal composition ratio, and an estimated packing density of 0.44 nm² per lipid headgroup. Branched, 25-kDa PEI was modified with amine reactive NHS-PEG (5 kDa) at a molar ratio of 5:1. Sulfhydryl binding sites were introduced via Trauts reagent onto the PEG-PEI. Analysis with Ellmans reagent indicated that an average of 9.6±3.7 (n=3) sulfhydryl groups/PEG-PEI were introduced during the thiolation process. The resulting PEG-PEI-SH was covalently bound to maleimide groups on the lipid-coated microbubbles. As reflected in the distributions in FIGS. 3A-3B, polymer grafting onto the microbubble surface did not significantly change the size distribution. The median diameter was 5.1±0.3 μm by volume and 4.1±0.2 μm by number (n=3). Such a size range may be useful for small animal imaging and therapy as well as human patient imaging and therapy.

The conjugation procedure was confirmed by coupling fluorescent F-PEG-PEI-SH to the microbubble surface and directly observing the microbubbles with fluorescence microscopy. In particular, fluorescent PEG-PEI-SH polymers were made utilizing amine reactive 5-carboxyfluorescein succinimidyl ester (NHS-Fluorescein). PEG-PEI-SH polymers were dissolved in PBS buffer (pH 8.4) to 10 mg/mL. NHS-Fluorescein was dissolved to 10 mg/mL in DMF and added drop-wise to the PEG-PEI-SH solution while rigorously mixing at molar ratio of 5:1. The solution was reacted for an hour and dialyzed for 48 hours using dialysis tubing with a 4-6 kDa MWCO. The resulting solution was subsequently freeze-dried for 48 hours to obtain the fluorescently labeled F-PEG-PEI-SH polymers.

FIGS. 4A-4B show bright field and fluorescence images, respectively, of microbubbles loaded with F-PEG-PEI-SH. The bright field image shows the presence of the gas core, as evidenced by the strong optical contrast, spherical shape and diffraction pattern. The presence of F-PEG-PEI-SH was observed on all microbubbles in epi-fluorescence mode. Some microbubbles exhibited surface folds and projections. No fluorescence was observed when using control microbubbles without maleimide, or when the maleimide was blocked using a molar excess of L-cysteine (data not shown).

The binding kinetics of PEG-PEI-SH to the maleimide microbubbles was determined using flow cytometry. Median fluorescent intensity (MFI) values of the microbubble sample were recorded before and after the addition of F-PEG-PEI-SH polymer. A gating technique was used to identify regions on the density-scatter plot corresponding to specific size ranges. Separate gating was performed using these regions to measure the MFI of microbubbles at 1-2, 4-5, and 6-8 μm diameters. Experiments were performed starting with 200 μL of size-selected maleimide bubbles (1×10⁹ MB/mL) per sample. 2 μL was taken for each measurement at each time-point and diluted in 100 μL of PBS. F-PEG-PEI-SH was added at a 10:1 PEI:maleimide molar ratio and briefly vortexed. MFI measurements were performed on each sample over 48 hours. Control experiments to determine non-specific binding of the polymer to the microbubble shell were performed by blocking the maleimide reaction with 1.000-fold molar excess of L-cysteine.

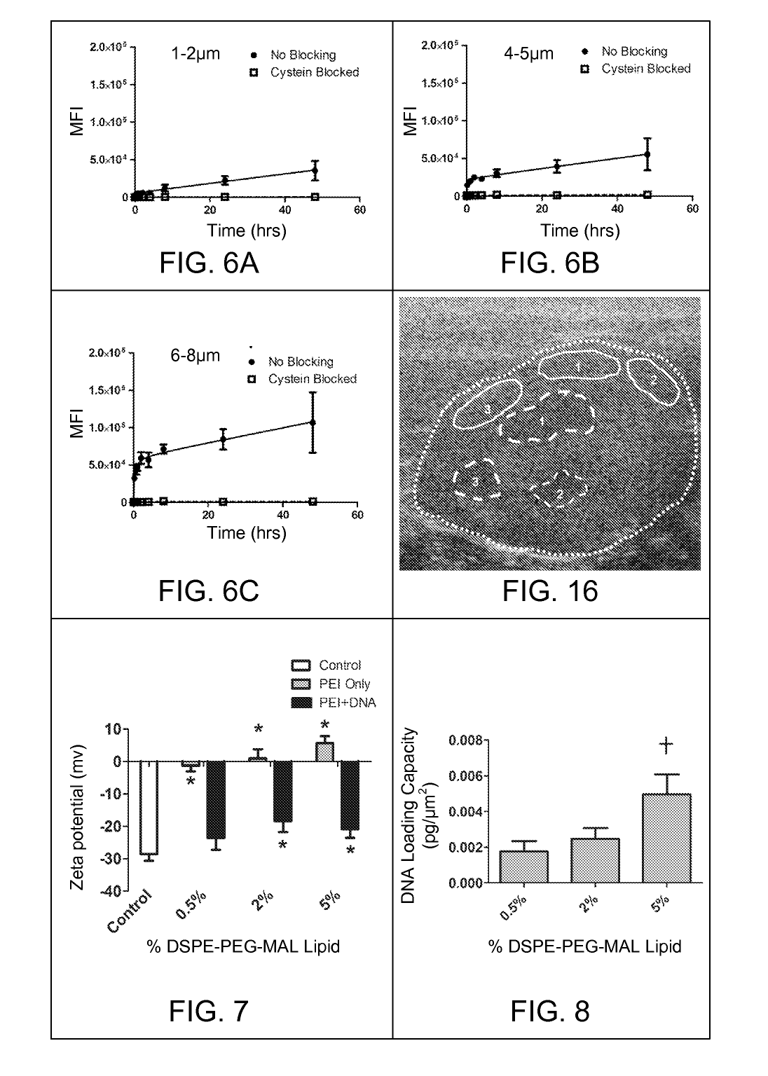

FIG. 5 is a density scatter plot of MFI per microbubble over 48 hours while FIGS. 6A-6C show the MFI versus time for gated regions B, C, and D, respectively, in FIG. 5 and corresponding to different microbubble size regions. Fluorescent readings were taken after mixing the maleimide-bearing microbubbles with and without blocking of the maleimide group with L-cysteine. The F-PEG-PEI-SH rapidly bound to the maleimide linkers on the microbubble shell within the first 3 hours, followed by a slower binding phase over the remaining 48 hours. The data was fit to a total binding saturation model to describe the trend for each microbubble size class (see Table 1). A total binding-saturation model for the MFI curves can be described by:

$$MFI = \frac{{B}_{ma x} * t}{{K}_{d} + t} + X * t + B , \qquad ( 1 )$$

wherein Bₘₐₓ is the total maximum specific binding (R.U.), t is time (hours), K_{d} is the equilibrium binding constant (hours), X is a non-specific binding term (R.U./hour) and B is the initial baseline MFI prior to F-PEG-PEI-SH incubation. The model assumes maleimide is the limiting reagent. The maximum specific binding (Bₘₐₓ), time to reach maximum binding and degree of nonspecific binding (X) both increased with microbubble size. No trend was observed for the equilibrium constant K_{d}. These results show that the 48-hour incubation was enough to complete the fast binding phase, as defined by the model.

TABLE 1
PEI Binding Kinetics to Microbubble Surface

| | | | Time to | | |
|---|---|---|---|---|---|
| Microbubble | Bₘₐₓ | K_{d} | Bₘₐₓ | X | B |
| Size Range | (R.U.) | (hours) | (hours) | (R.U./hour) | (R.U.) |
| 1-2 μm | 5,700 | 0.26 | 1.2 | 640 | 280 |
| 4-5 μm | 23,000 | 0.14 | 1.8 | 680 | 340 |
| 6-8 μm | 59,000 | 0.23 | 3.1 | 1,000 | 1,200 |
| 1-2 μm Blocked | N/A | N/A | N/A | 9.4 | 310 |
| 4-5 μm Blocked | N/A | N/A | N/A | 19 | 1,100 |
| 6-8 μm Blocked | N/A | N/A | N/A | 4.8 | 490 |

In order to demonstrate that PEI attachment was due to a stable thioether bond, rather than a nonspecific interaction, the maleimide linker was blocked with L-cysteine prior to mixing with F-PEG-PEI-SH. In this case, the MFI did not increase above the baseline value at any time-point (P>0.05), indicating the absence of electrostatic or other nonspecific adsorption of PEI to the microbubbles. The use of a covalent thioether bond was expected to aid in stabilizing the microbubble/PEI/DNA complex for in vivo experiments.

The DNA loading capacity of the PEI-microbubbles was measured using salmon sperm DNA. Salmon sperm DNA was dispersed to 1 mg/mL by probe sonication for 5 minutes. 500 μL containing 109 PEI-loaded microbubbles was added drop-wise to 500 μL of DNA solution while gently mixing. The DNA was allowed to electrostatically couple to the polymer-coated microbubbles while gently mixing for 1 hour. The microbubbles were then concentrated by centrifugation and washed 3 times in a syringe (90 RCF; 1 min; 10 mL washing volume) to remove unbound DNA. The concentration and size distribution of remaining microbubbles was measured to determine the maximum surface area available for DNA loading, assuming the microbubbles were spheres. The sample was then heated to 65° C. for several hours and briefly bath sonicated until the bubbles were destroyed, evidenced by the solution becoming clear. The amount of DNA in the sample was measured by UV absorbance at 260 nm using a spectrophotometer.

The surface charge of microbubbles loaded with PEG-PEI-SH was measured for varying maleimide concentrations and compared to control microbubbles without polymer. A graph of zeta potential (mv) for various maleimide concentrations is shown in FIG. 7. Zeta potential analysis shows a significant change in the surface chemistry after addition of the PEI polymer. The charge was initially negative owing to the phosphate on the PEG-lipids and the maleimide groups (5 mol %). Following addition of cationic PEG-PEI-SH, the charge was neutralized for 0.5 and 2.0 mol % maleimide and reversed in sign to become cationic at 5 mol % maleimide. All PEI-loaded groups showed significant increases in zeta potential compared to control (P<0.0001, n=3 per group). Addition of DNA to the PEI-loaded microbubbles reversed the surface charge back to negative values for every group. Covalent attachment prevented PEI from simply desorbing from the surface due to interactions with DNA. This reversal in surface charge therefore indicated that PEI was successful in sequestering DNA from the bulk through electrostatic interactions.

FIG. 8 shows the total DNA loading capacity per unit surface area of the PEI-microbubbles. The loading capacity increased in proportion with maleimide-lipid concentration. This result was consistent with the zeta potential measurements described above, i.e., more maleimide led to greater PEI deposition, which in turn led to greater DNA loading. A high DNA loading capacity of 0.005 pg/μm² was achieved. Thus, PEI loading of the microbubbles (and thereby DNA loading of the polyplexes on the microbubbles) can be controlled by modulating the maleimide content of the microbubble shell.

A theoretical loading efficiency can be calculated based on the available maleimide groups on the microbubble surface with a few reasonable assumptions. Based on the molar composition of the lipid and a 0.44 nm² lipid head cross-sectional area, the estimated surface density of maleimide groups is 1.14×10⁵ molecules/μm². Assuming a PEI:maleimide ratio of 1:10 (based on the number of measured sulfhydryl groups per PEI), and complete saturation of all PEI amine groups with DNA phosphate groups (for example, 580 amine groups per PEG-PEI), the estimated maximum loading density of DNA onto the microbubble surface is 0.004 pg/μm² for 5% maleimide, which is close to the measured value of 0.005±0.001 pg/μm² above.

In one or more embodiments, ligands can be conjugated to the microbubble surface in order to facilitate specific adhesion to the tumor vasculature expressing the target receptor molecule. For example, an antibody can be used to target VEGFR2, or a thiolated, cyclic arginine-glycine-aspartic acid (RGD) peptide can be used to target αᵥβ₃ integrin. Synthetic peptides may result in reduced batch-to-batch variation, less immunogenicity, better control of ligand orientation and higher density on the microbubble surface. Solution-phase conjugation chemistry (maleimide-thiol) can be performed on microbubbles following fabrication and size isolation. The targeting ligand can be added to the microbubble suspension and allowed to incubate for 2 hours at room temperature under mild stirring using a benchtop rotator, which keeps the microbubbles uniformly distributed throughout a capped syringe. The maleimide group on the microbubble shell reacts with the thiol group of the targeting ligand in the deoxygenated, PFB-saturated aqueous solution. After coupling, unreacted maleimide can be quenched by reduction with 2 mmol/L β-mercaptoethanol for 30 minutes at room temperature.

Ligand conjugation to the microbubble surface can be confirmed by flow cytometry and fluorescence microscopy using fluorescein isothiocyanate (FITC) modified ligand and high pressure liquid chromatography (HPLC) using the native ligand. The fluorescence assays can provide a rapid, high-throughput means of assessing ligand conjugation. FITC tagging of the peptides/antibodies can be accomplished by reacting FITC-NHS with primary amines present on the ligand. The fluorescent ligand can be characterized by HPLC. For HPLC, FITC-ligand can be eluted from a C18 column by slowing changing the composition of acetonitrile and water in the mobile phase. Absorption can be measured at 220 nm and 494 nm to confirm FITC conjugation. Purified FITC-ligand conjugate can be collected, analyzed by mass spectrometry and used for flow cytometry. Flow cytometry can provide a saturation curve (MFI vs. mg-ligand/μm²-microbubble) for each ligand to determine the appropriate ratio of ligand to microbubble surface area. Fluorescence microscopy can be used to directly image heterogeneity of the ligand over the microbubble surface. HPLC analysis can provide a final determination of average ligand surface density on the microbubble surface.

For example, PEG groups of the polyplex and/or the microbubble shell can be coupled to targeted ligands, such as, but not limited to RGD, which can bind to the αᵥβ₃ integrin receptor on endothelial cells to increase contact between the microbubble and the cell membrane. Transfection efficiency may thus be increased by targeting vasculature with the microbubbles labeled with RGD peptide or an anti-VEGFR2 antibody. Such microbubbles may be employed in a therapeutic use, such as by targeting AKT1 gene using shRNA AKT1 polyplexes in conjunction with VEGF inhibition. The expression of an angiogenic biomarker, αᵥβ₃ integrin, can be quantified using ultrasound molecular imaging with targeted microbubbles. RGD-labeled microbubbles can be injected via the femoral vein to target the angiogenic marker αᵥβ₃ integrin. B-mode imaging allowed positioning of the ultrasound transducer over the tumor in a region of interest, shown in FIGS. 10A-10B for control microbubbles (i.e., untargeted) and targeted microbubbles, respectively. The control or targeted microbubbles were injected intravenously and allowed to circulate for a 12-min dwell time, during which time targeted microbubbles adhere to the tumor vasculature. Contrast intensity within the region of interest was determined in each frame.

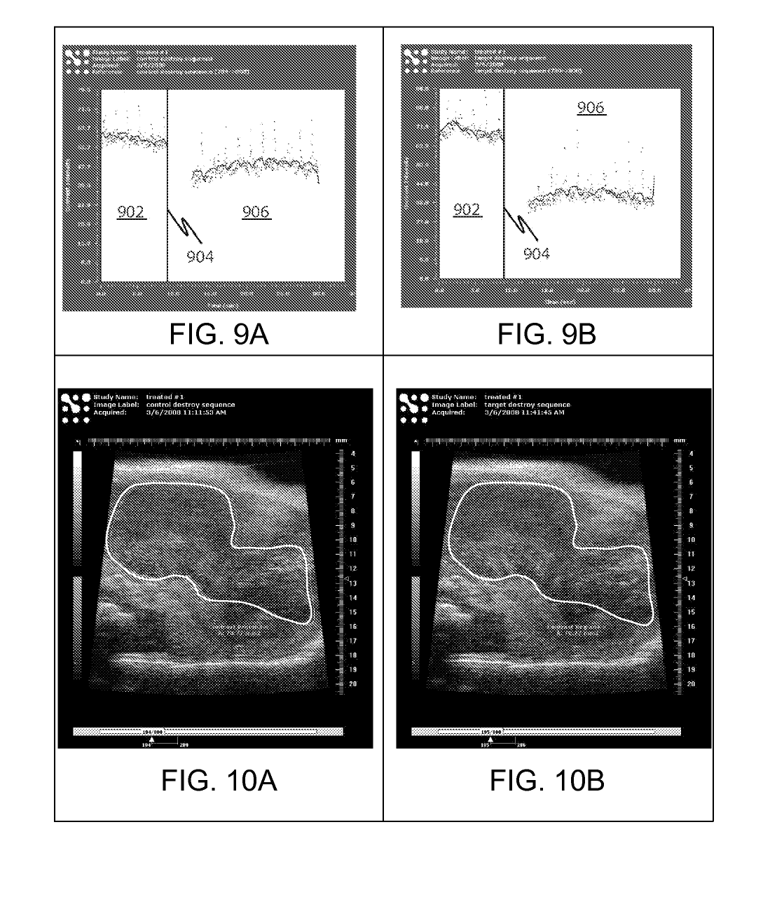

Time intensity plots for the control microbubbles and the targeted microbubbles are shown in FIGS. 9A-9B, respectively. During a period 902 prior to application of the destruction pulse at 904, the signal is present from bound microbubbles, free microbubbles and tissue motion. At 904, a low-frequency, high-power pulse was used to fragment microbubbles in the field of view. A 4-sec reflow time was observed, after which the contrast intensity was again recorded during the period 906. During period 906, a subsequent plateau gives the signal from free microbubbles and tissue motion. The difference between before and after the destruction pulse gives the signal from just bound microbubbles. The difference between the control and targeted microbubbles gives a measure of specific versus nonspecific adhesion. Specificity was clearly indicated with a 20 dB increase for targeted microbubbles versus control microbubbles.

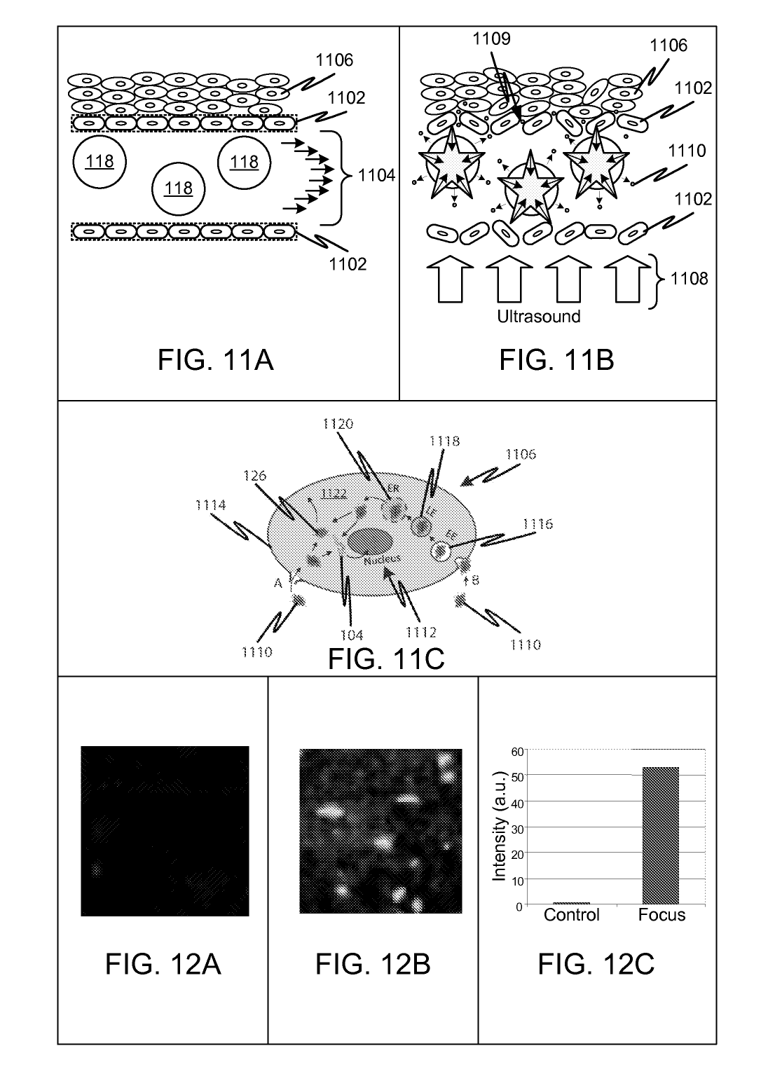

As discussed above, the polyplex-loaded microbubbles 118 can be used to transfect plasmid DNA 104 carried by the microbubble to cells within a patient. Referring now to FIG. 11A, a schematic diagram of a portion of the vasculature within a region of interest of the patient is shown. For example, the vasculature may be that of a cancerous tumor within the patient. Polyplex-loaded microbubbles 118 can be injected intravenously and allowed to circulate through the blood stream 1104. Vascular endothelial cells 1102 border the blood flow and separate the blood flow 1104 as well as the microbubbles 118 therein from desired cells 1106 to be transfected, e.g., tumor cells.

High-pressure, low-frequency ultrasound can be used to focus the effects of microbubble interactions on cells, such that gene transfection is predominantly contained within the region of the applied ultrasound. Transfection via microbubble destruction may predominantly occur in the focal region of the ultrasound transducer when a radiation force pulse is applied, while transfection in areas outside of the focal region is significantly less. FIGS. 12A-12C illustrate this principle using CMV-promoted plasmid DNA encoding green fluorescent protein (GFP) for the transfection of plated A375 human melanoma cells. In FIG. 12A, a control sample was outside of the focal region of the ultrasound, while in FIG. 12B, the region of interest was within the focal region of the high-pressure, low-frequency ultrasound. As is evident from the graph in FIG. 12C, a significant increase in fluorescence intensity due to the transfection of DNA encoding GFP occurs for the application of ultrasound. By exploiting the ability of ultrasound to precisely control microbubble distribution, highly specific tissue targeting of proteins and plasmids to the heart, tumors, and other tissues can be achieved.

By applying high-pressure, low-frequency ultrasound 1108 to the region of interest, the microbubbles 118 in the region of interest, whether bound to target sites of the vascular cells 1102 or flowing in the blood flow 1104 through the region of interest, are destroyed, as shown in FIG. 11B. In particular, the ultrasound 1108 causes microbubbles 118 within the region of interest to undergo inertial cavitation. Oscillations of the gas core of the microbubble 118 induced by the ultrasound 1108 can create pores 1109 (i.e., via sonoporation) between the vascular cells 1102 and surrounding cell membranes, e.g., of cells 1106, through which genetic material may pass to enter the cell cytoplasm. In addition to the permeation of the endothelial lining 1102, microbubble fragmentation caused by the ultrasound 1108 allows for releases of polyplexes/lipids 1110 and the accompanying genetic payload.

Polyplexes and/or DNA can enter into the desired cells via two mechanisms. Physical disruption of the cell membrane 1114, for example, due to the sonoporation, can allow passive entry of the polyplex 1110 into the cytoplasm 1122. Once inside the cell membrane, the polyplex 1110 may dissociate into plasmid DNA 104 and PEI polymer 126. The plasmid DNA 104 may subsequently enter the cell nucleus 1112. Alternatively or additionally, the polyplex 1110 can breach the cell membrane 1114 via enhanced clatherin-mediated endocytotic uptake. In such a mechanism, the PEI facilitates interaction with the cell membrane 1114, such that the polyplex 1110 is taken up into an early endosome 1116. The early endosome 1116 is then trafficked into late endosomes 1118 or lysosomal compartments. Osmotic swelling caused by PEI may result in endosomal rupture at 1120 via a proton-sponge effect, thereby allowing the polyplex 1110 entry into the cytoplasm 1122. Plasmid DNA 104 dissociates from the PEI/lipid vector 126 and enters the nucleus 1112 of the cell 1106 whereby the genes of the DNA 104 can be expressed. DNA plasmid 104 is thus able to extravasate into cells 1106 in vivo through a combined mechanism of microbubble-induced sonoporation and PEI-enhanced extra/intra-cellular trafficking.

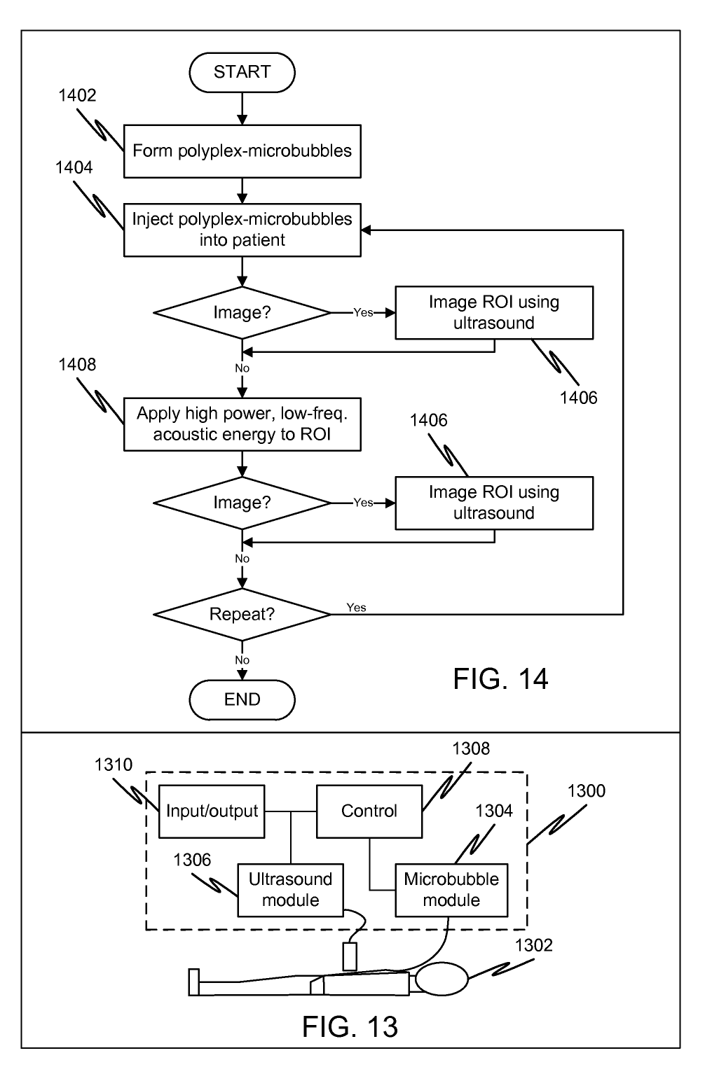

Referring to FIG. 13, a system for gene transfection using polyplex-loaded microbubbles is shown. The system 1300 may be used for gene transfection in a patient 1302, which may be a human or animal, as part of treatment (i.e., cancer therapy) or study. System 1300 can include a microbubble module 1304. Microbubble module 1304 can be configured to provide and/or inject the polyplex-loaded microbubbles described herein to the patient 1302. Microbubble module 1304 can also be configured to produce the polyplex-loaded microbubbles prior to injection, for example, from stock polymer materials and DNA. For example, the microbubble module 1304 can include a syringe containing a suspension of polyplex-loaded microbubbles and a syringe pump for intravenously injecting the syringe contents into the patient 1302 at a controlled rate.

The system can further include an ultrasound module 1306. The ultrasound module 1306 can have an input/output unit 1310 coupled thereto. The input/output unit 1310 can include, for example, a display for conveying ultrasound image data to an operator. The input/output unit 1310 can also be configured to accept inputs from the operator, for example, with regard to location of ultrasound focus, intensity of ultrasound, and/or timing of destruction pulse. The system 1300 can also have a control module 1308 coupled thereto in order to control operation of the ultrasound module 1308 and/or the microbubble module 1304.

The ultrasound module 1306 can be configured to obtain ultrasound images of a region of interest in patient 1302 during a first mode of operation. During this first mode of operation, polyplex-loaded microbubbles may or may not be flowing through the region of interest of the patient. If microbubbles are in the region of interest, the ultrasound applied during the first mode of operation may be of such a magnitude and/or frequency such that the microbubbles in the region of interest are not destroyed. Thus, the region of interest and the microbubbles therein may be imaged during the first mode of operation of the ultrasound module 1306.

The ultrasound module 1306 can also have a second mode of operation different from the first mode. In the second mode of operation, a high-intensity, low-frequency acoustic energy can be applied to the region of interest to thereby destroy microbubbles therein and allow gene transfection. This second mode of operation may occur simultaneously with the first mode, i.e., that the high-intensity, low-frequency acoustic energy happens concurrently with the imaging. Additionally or alternatively, the second mode of operation may occur at a time period between first modes of operation. For example, the second mode of operation may be a relatively short burst of high-intensity, low-frequency acoustic energy between otherwise continuous ultrasound imaging periods.

Although illustrated as separate components in FIG. 13, one or more of the units and modules of system 1300 can be combined together to form other units or modules. In addition, the separately illustrated components of FIG. 13 may be part of a single module or unit. Alternatively or additionally, one or more of the illustrated components of FIG. 13 may be embodied as multiple units or modules. For example, a separate ultrasound module may be provided for the functions performed by ultrasound module 1308, i.e., a first ultrasound module dedicated to imaging and a second ultrasound module dedicated to applying the high-intensity, low-frequency pulse for microbubble destruction. In another example, a separate microbubble module may be provided for the functions performed by microbubble module 1304, i.e., a first microbubble module for forming the polyplex-loaded microbubbles and a second microbubble modules for injecting the polyplex-loaded microbubbles. Other configurations for the system 1300 are also possible according to one or more contemplated embodiments.

Referring to FIG. 14, a flow diagram of a method of gene transfection using polyplex-loaded microbubbles is shown. The method begins at 1402 where polyplex-loaded microbubbles are formed. For example, the polyplex-loaded microbubbles can be formed according to the method of FIG. 2 and as described herein. At 1404, the polyplex-loaded microbubbles can be introduced into the bloodstream of the patient. For example, the microbubbles can be dispersed in solution so as to form a suspension and injected into the bloodstream of the patient. Such injection may be done manually, for example, by a physician or other caregiver, or automatically, for example, by a syringe pump. Alternatively, the microbubbles can be directly introduced into the desired tissue vasculature.

After sufficient time for the circulating microbubbles to reach the vasculature in the desired region of interest, the method can optionally proceed to 1406, where the region of interest and the microbubbles therein are imaged using ultrasound. The ultrasound may be of sufficient power and/or frequency such that microbubbles in the region of interest are not destroyed during the imaging. During this time, the microbubbles may also serve as ultrasound contrast agents to enhance imaging of the region of interest. After 1404 (or after optional step 1406), high-intensity, low-frequency acoustic energy (e.g., ultrasound) can be applied to the region of interest such that microbubbles therein undergo inertial cavitation and fragmentation. Polyplexes from the fragmented microbubbles can thus enter cells in or bordering the region of interest. Imaging 1406 may also be performed after application of the high-intensity, low-frequency acoustic energy. The process may be repeated any number of times with the same or different polyplex-microbubbles in order to transfect additional and/or different DNA to cells in the region of interest.

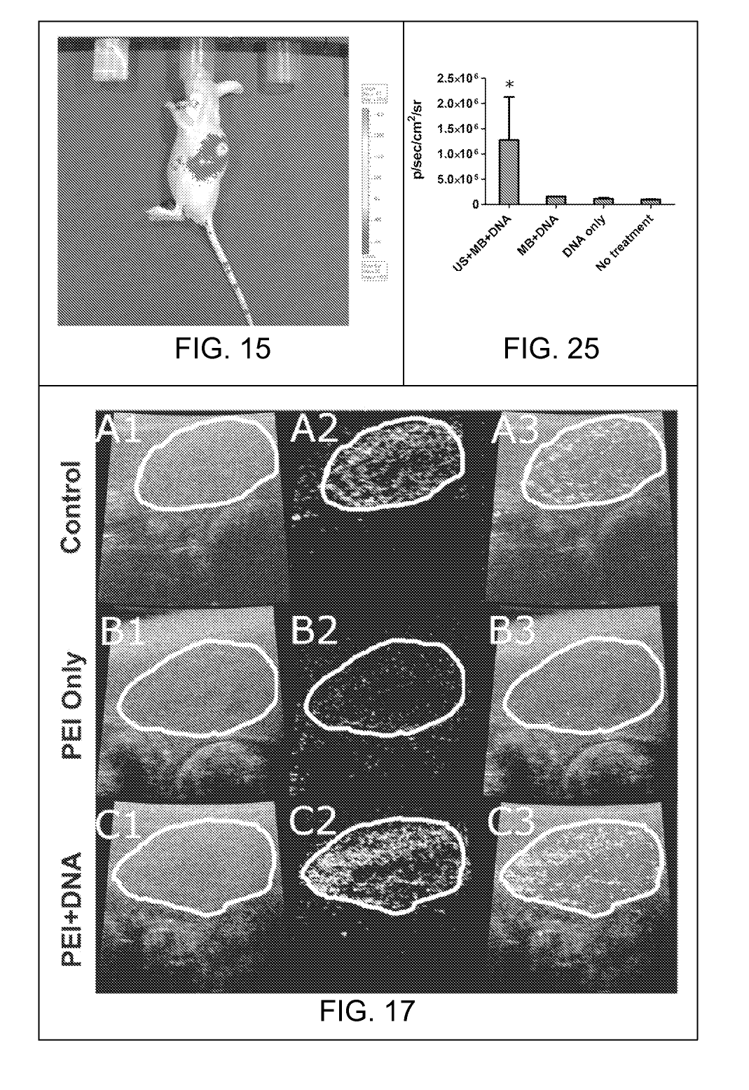

Transfection of tumors can be demonstrated using luciferase plasmid-bearing microbubbles and sonoporation. Mice bearing neuroblastoma xenograft tumors implanted in the left kidney were injected with microbubbles coated with plasmid DNA encoding the cytomegalovirus (CMV) promoter and luciferase enzyme (in a single DNA layer). Following tail vein injection of the microbubbles, the tumor was insonified intermittently at 1 MHz, 2.0 W/cm² with a 10% duty cycle for 5 second intervals. Gene expression was observed 2 days later as bioluminescence after luciferin injection using a fluorescence imaging system and is shown in FIG. 15. Strong luminescence can be seen coming from the transducer focal point over the tumor in FIG. 15.

Contrast-enhanced ultrasound persistence studies were performed in female CD-1 mice 4-6 weeks of age. Mice were anesthetized using 1-2% isofluorane and placed on a mouse handling table, and the heart rate, respiratory rate and temperature were monitored. Mice were kept under anesthesia for the duration of the experiment. After the mouse was completely anesthetized, the tail vein was catheterized using a modified 27-gauge, ½-inch butterfly catheter. Prior to catheterization, the tubing was removed and replaced with smaller 27-gauge Tygon® tubing (0.015″ inner-diameter). The mouse was shaved in the kidney region.

A small animal ultrasound imaging scanner with a 30-MHz imaging transducer was placed over the kidney of the mouse and coupled using ultrasound transmission gel. A bolus injection of 50 μL of microbubble solution (2.5×10⁷ microbubbles/bolus) was injected while imaging continuously at 16 frames per second (100% power setting). Respiratory gating was used to synchronize data acquisition with the mouse respiratory cycle, in order to reduce motion artifact during image analysis. Respiratory gating lowered the effective acquisition rate to 2 frames per second. Ultrasound imaging was performed between 5 and 20 minutes following injection of the microbubble suspension.

Mice were injected with control, PEI-loaded and DNA/PEI-loaded microbubbles using sonicated salmon sperm DNA. Each mouse was given three randomized injections per imaging session, with 20 minutes between start points of the injections, and then removed from anesthesia. Experiments were repeated in triplicate at 0.5 mol %, 2 mol %, and 5 mol % DSPE-PEG-Mal compositions. Control microbubbles contained 0% DSPE-PEG-Mal and 10% DSPE-PEG2k.

Multiple regions of interest (ROI) in the kidney were selected, as shown in FIG. 16. Three ROIs (solid line) in the upper portion of the kidney encompassing the cortex region were used to evaluate the change in average video pixel intensity over time caused by the presence of microbubbles (time-intensity curves; TICs). The signals from the three ROI's were averaged to obtain a final TIC. Three additional ROIs (dashed line) were selected to encompass hypoechoic areas where the medulla and larger blood vessels were more prominent. A motion analysis algorithm using normalized two-dimensional cross correlation was implemented to evaluate the signal fluctuation caused by circulating microbubbles. The motion analysis algorithm was used to generate a time-fluctuation curve (TFC), which was used to distinguish between freely circulating and adherent microbubbles. The signals from the three regions of interest were averaged to obtain the final TFC.

Plasmid DNA was isolated and was encoded for the bioluminescent protein luciferase. Luciferase plasmid DNA was dissolved in nuclease free water to 2 mg/mL. UV/VIS spectrometry was used to determine the DNA concentration. Tumors were formed in female nude NCR mice injected with a SKNEP-1 human cancer cell line. For each mouse, 106 cells were injected directly into the left kidney through a small incision in the left flank. Tumors were allowed to develop for 5 weeks and were palpated every week to determine size. Five weeks after implantation, the mice were transfected with PEI-microbubbles mixed with the plasmid DNA (108 microbubbles with 500 μg DNA in total of 400 μL injection volume).

Each mouse was anesthetized using ketamine/xylazine, and the tail vein was catheterized using a custom 27-gauge, ½-inch butterfly catheter. A therapeutic ultrasound machine with a 2 cm diameter soundhead was placed over the tumor region. The polyplex-microbubble suspension was injected slowly (e.g., at a rate of 0.2 mL/min) while applying continuous ultrasound at 1 MHz, 1 W/cm², and 10% duty cycle. Ultrasound was administered for a total of 10 minutes following the start of injection of the microbubble-DNA solution. Ultrasound was manually turned off every 5 seconds, for 5 seconds duration, to allow replenishment of new microbubbles into the tumor vasculature. After the mouse regained consciousness, it was returned to its cage. Bioluminescence was measured in vivo at 2 days post transfection, 5 minutes after a 100 μL intraperitoneal injection of D-Luciferin. All images were taken with a bioluminescent in vivo imaging system using 1 minute exposure times and medium binning.

A group of mice were sacrificed immediately after in vivo luciferase imaging, and their tissues were excised to test the specificity of luciferase expression in the tumor. 0.2 grams of tissue (tumor and heart) was collected, weighed and homogenized on ice using a tissue homogenizer in 800 mL of passive lysis buffer, followed by centrifugation. 40 mL of the supernatant was added to 100 mL of luciferase assay reagent and read in a luminometer. The relative luciferase units were normalized to the tumor weight. Students' t-tests were performed to evaluate significant differences between treated and control groups.

DNA transfections using a luciferase plasmid were performed on SKNEP-1 tumor-bearing mice using the 5 mol % maleimide polyplex-microbubbles. No adverse effects in the NCR nude mice were observed after anesthesia recovery using 400-4 injection volumes of the microbubble formulations. In vivo bioluminescence imaging at 48 hours post-transfection showed site-specific luciferase expression in the abdominal area flanking the kidney where the tumor was implanted and the ultrasound transducer was applied (see FIG. 24A). The photon flux from the tumor area was measured to be over 10-fold higher than the baseline signal from untreated mice (see FIG. 25). The luciferase expression measured ex vivo was over 40-fold higher in tumor tissue than in heart tissue in animals that received DNA/PEI-microbubbles and ultrasound (see FIG. 26). The ultrasound transducer was placed in the lower abdominal region such that the heart tissue was not exposed to ultrasound. Heart tissue was used as an internal control to demonstrate lack of luciferase expression where ultrasound was not applied. No bioluminescence was detected above background in the mice exposed to polyplex-microbubbles without ultrasound (see FIG. 24B). As FIGS. 24A-24C, 25, and 26 suggest, both microbubbles and ultrasound application is necessary to transfect the tumors, and the transfection was isolated to the tissue exposed to ultrasound.

The circulation persistence of the PEI-coated microbubbles with and without DNA loading was evaluated in vivo using high frequency ultrasound. A bolus of 2.5×10⁷ microbubbles was injected intravenously via the tail vein while monitoring the contrast signal in the mouse kidney. No adverse effects from the microbubble injections were observed in the CD-1 mice after anesthesia recovery. FIG. 17 shows grayscale B-mode ultrasound images (column 1), contrast detection (column 2), and B-mode/contrast overlays (column 3) shortly after microbubble injection when the signal intensity had reached the maximum level. Contrast was detected as an increased scattering signal following reference subtraction using pre-contrast images.

Panel A in FIG. 17 shows a typical ultrasound image snapshot following a bolus injection of control microbubbles. The contrast was distributed throughout the kidney region (denoted by the white border) with greater intensity in the highly vascularized cortex. Panel B in FIG. 17 shows a representative image for 5% maleimide PEI-microbubbles without DNA loading. The contrast signal was much less conspicuous. Panel C in FIG. 17 shows an image for 5% maleimide PEI-microbubbles loaded with DNA. The contrast was much higher for DNA/PEI-microbubbles as compared to PEI-microbubbles, and the contrast intensity and spatial distribution were similar to those for control microbubbles.

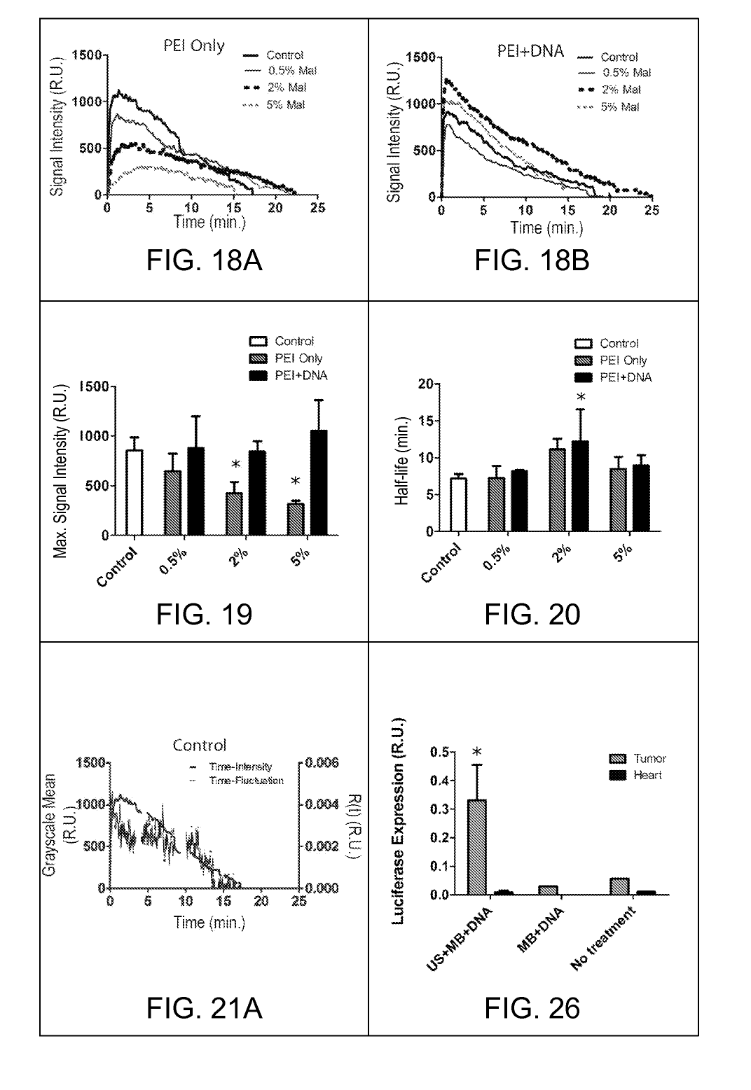

FIGS. 18A-18B show typical time intensity curves (TICs) generated after a bolus injection (2.5×10⁷ microbubbles) for PEI-microbubbles and polyplex-microbubbles, respectively, with varying degrees of maleimide-lipid in the microbubble shell. The shapes of the TICs are noticeably different for PEI-microbubbles as compared to polyplex-microbubbles or control. PEI-microbubbles tended to have a lower maximum signal intensity, as shown in FIG. 19, and slower “wash-in” phase. This effect increased with increasing PEI content (equivalently, maleimide content). Polyplex-microbubbles, on the other hand, exhibited similar TICs to control.

The TICs were fit to a single-compartment model, which allowed determination of the maximum intensity and half-life of the total contrast signal. The mean maximum signal intensity was significantly less for PEI-microbubbles compared to DNA/PEI-microbubbles or control for 2% and 5% maleimide, but not for 0.5% maleimide in the microbubble shell, as shown in FIG. 19. The mean maximum intensity for DNA/PEI-microbubbles was statistically equal to that for control microbubbles. Despite the lower signal intensity for PEI-microbubbles, the mean half-life was not statistically different between any of the groups, as shown in FIG. 20. Additionally, less “speckling” was observed in the ultrasound videos for PEI-microbubbles compared to the other groups, suggesting that PEI-microbubbles adhere nonspecifically, through electrostatic interactions, to the vascular endothelium. The reduced intensity may result from adhesion to vasculature upstream of the kidney (e.g., in the tail vein and pulmonary capillary bed) leading to loss of microbubbles prior to entering the kidney for imaging. The long half-life may be explained by the relatively slow dissolution of adherent microbubbles.

A two-compartment model can be used to distinguish freely circulating microbubbles from adherent, non-circulating ones using the TICs and time-fluctuation curves (TFCs), which can be obtained using a normalized cross-correlation algorithm. Frame-by-frame decorrelation was used to detect changes in the speckle pattern in the region of interest in order to measure the fluctuation of the signal caused by circulating microbubbles. Each ultrasound video was processed to obtain a TFC and TIC.

FIGS. 21A-21G shows examples of the TICs overlaid with the corresponding TFCs. The signal persistence time of the TFCs and TICs appeared to be similar for the control, indicating that circulating microbubbles were the primary contributor to the overall signal enhancement (FIG. 21A). For PEI-microbubbles, however, there was a noticeable difference between the TICs and the TFCs. The TFCs rapidly decreased to baseline (FIG. 21D), or were non-existent (FIG. 21F), while the TICs exhibited prolonged persistence. This discrepancy may be due to the cationic charge of PEI-microbubbles, which caused them to adhere through electrostatic interactions with the negatively charged glycocalyx on the vascular endothelium.

The polyplex-microbubbles (i.e., DNA-loaded PEI-microbubbles) experienced behavior somewhat in between that of control and PEI-microbubbles (see FIGS. 21C, 21E, and 21G). For these microbubbles, the TFCs deviated from the TICs, but not to the same extent as for the PEI-microbubbles. For both DNA-loaded and unloaded PEI-microbubbles, the difference between TFCs and TICs increased with increasing maleimide, showing that this effect can be modulated by microbubble surface chemistry. The TICs and TFCs were fit to a two-compartment model. Compartment 1 contains freely circulating microbubbles, while compartment 2 contains microbubbles adherent to the kidney vasculature (i.e., non-circulating), which slowly dissolve away. Table 2 shows a summary of model coefficients for each group.

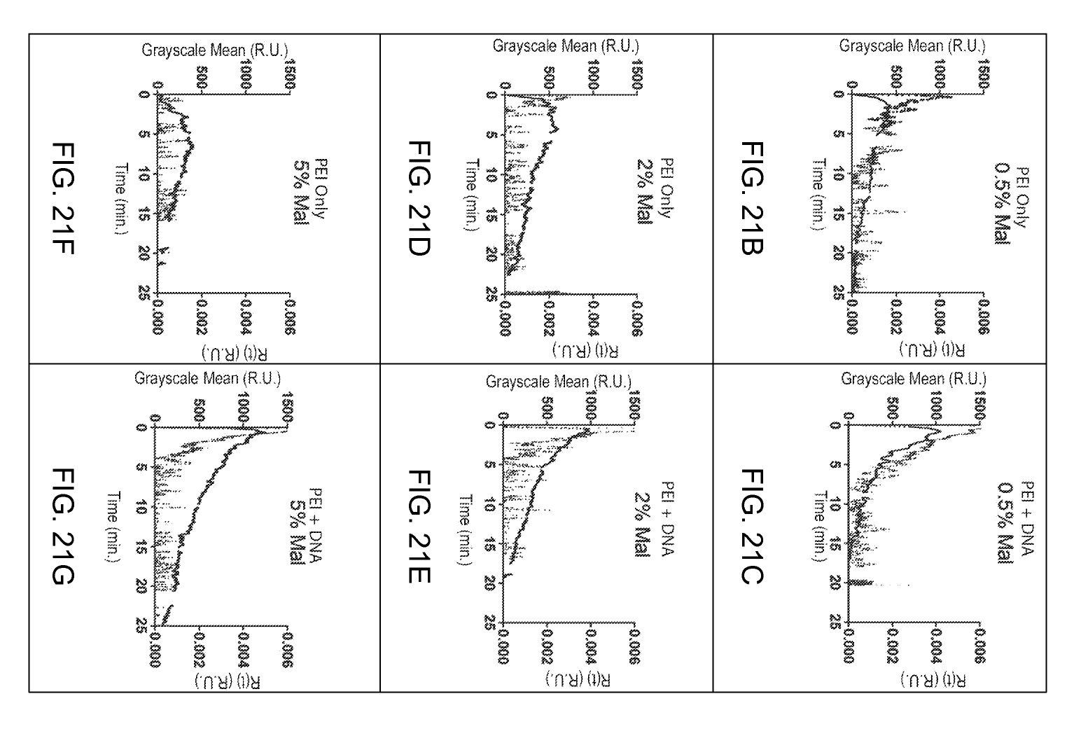

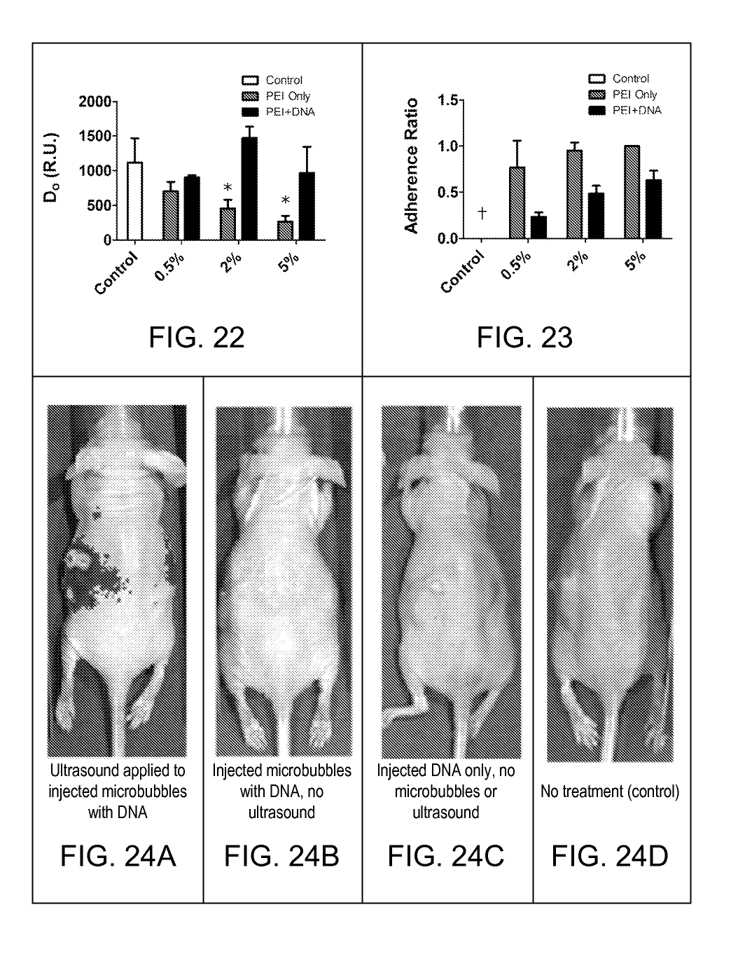

The Dₒ coefficient describes the total contrast agent delivered to the kidney and is closely related to the maximum signal intensity of the TIC, as shown in FIG. 22. The rate constants (k₁-k₄) describe the influx or efflux of contrast agent signal from compartments 1 and 2. The value of k₁, the influx rate of contrast agent into circulation from the bolus injection, was not significantly different between polymer-modified microbubbles and control. Variations may be due to the differences in bolus injection speed and in the heart and respiratory rates between animals. However, for 5% maleimide, the average k₁ value for polyplex-microbubbles was 12-fold higher than for PEI-microbubbles (P<0.05). The value of k₂, the elimination rate of contrast signal from circulation, was significantly lower for PEI-microbubbles (2% and 5% maleimide) as compared to the control (P<0.05). These trends in k₁ and k₂ suggest that PEI-microbubbles adherent upstream may be recirculating into the kidney.

TABLE 2
Exemplary coefficients for two-compartment model

| | D₀ | k₁ | k₂ | k₃ | k₄ |
|---|---|---|---|---|---|
| | (R.U.) | (min⁻¹) | (min⁻¹) | (min⁻¹) | (min⁻¹) |
| Control | 1100 ± 350 | 3.0 ± 2.5 | 0.10 ± 0.05 | 8.9 × 10⁻⁸ ± 7.3 × 10⁻⁸ | 0.04 ± 0.04 |
| 0.5% Mal | 700 ± 130 | 2.7 ± 1.4 | 0.04 ± 0.05 | 0.27 ± 0.21 | 0.15 ± 0.04 |
| No DNA | | | | | |
| 0.5% Mal | 900 ± 30 | 3.9 ± 0.2 | 0.14 ± 0.001 | 0.04 ± 0.01 | 0.07 ± 0.05 |
| DNA loaded | | | | | |
| 2% Mal | 460 ± 120 | 1.5 ± 0.5 | 0.01 ± 0.01 | 0.37 ± 0.29 | 0.10 ± 0.06 |
| No DNA | | | | | |
| 2% Mal | 1500 ± 160 | 1.5 ± 0.3 | 0.36 ± 0.18 | 0.31 ± 0.06 | 0.13 ± 0.09 |
| DNA loaded | | | | | |
| 5% Mal | 260 ± 85 | 0.4 ± 0.2 | ~0 | Inf. | 0.2 ± 0.08 |
| No DNA | | | | | |
| 5% Mal | 970 ± 380 | 4.7 ± 1.2 | 0.44 ± 0.25 | 0.68 ± 0.13 | 0.07 ± 0.05 |
| DNA loaded | | | | | |

PEI-microbubbles also showed an increase in the value of k₃, the rate at which microbubbles adhere to kidney vasculature, compared to control. The mean k₃ value for 2% maleimide PEI-microbubbles was higher than for the control, but not statistically significant (P=0.08). For the 5% maleimide PEI-microbubbles, no increase in the time-fluctuation signal was detected above baseline, which suggested that the cationic microbubbles were rapidly becoming adherent after entering the kidney (k₃→∞, k₂→0). No significant difference was observed for the dissolution rate of the adherent bubbles (k₄) for any maleimide concentration.

FIG. 23 shows the ratio of microbubbles that became adherent compared to those that remained freely circulating, as calculated from the k₂ and k₃ parameters. Control microbubbles have a very low adhesion ratio. On the other hand, adhesion was high for PEI-microbubbles and increased with increasing maleimide content. DNA loading onto the PEI-microbubbles to produce the polyplex-microbubbles decreased the adhesion ratio. However, some adhesion was observed at each maleimide concentration and increased with the maleimide content.

In summary, control microbubbles showed almost no adhesion and the resulting TIC was primarily from freely circulating microbubbles. As the amount of PEI conjugation increased, the signal from freely circulating microbubbles diminished and the signal from adherent bubbles became more prevalent. Loading of DNA onto the PEI-microbubbles improved the circulation profile at every maleimide concentration, although the half-life of the control microbubbles in circulation remained significantly greater (˜8 fold).

Regardless of whether they are freely circulating or adherent to the vasculature, the polyplex-microbubbles can persist on the order of tens of minutes. As compared to some nanocarriers, such as liposomes and filomicelles, which have reported circulation times on the order of hours to days, this may be a relatively short persistence time. However, on-demand and site-directed delivery offered by sonoporation precludes the need for enhanced permeability and retention (EPR) effect to target cells, and thus lessens restraints for a long-circulating carrier.

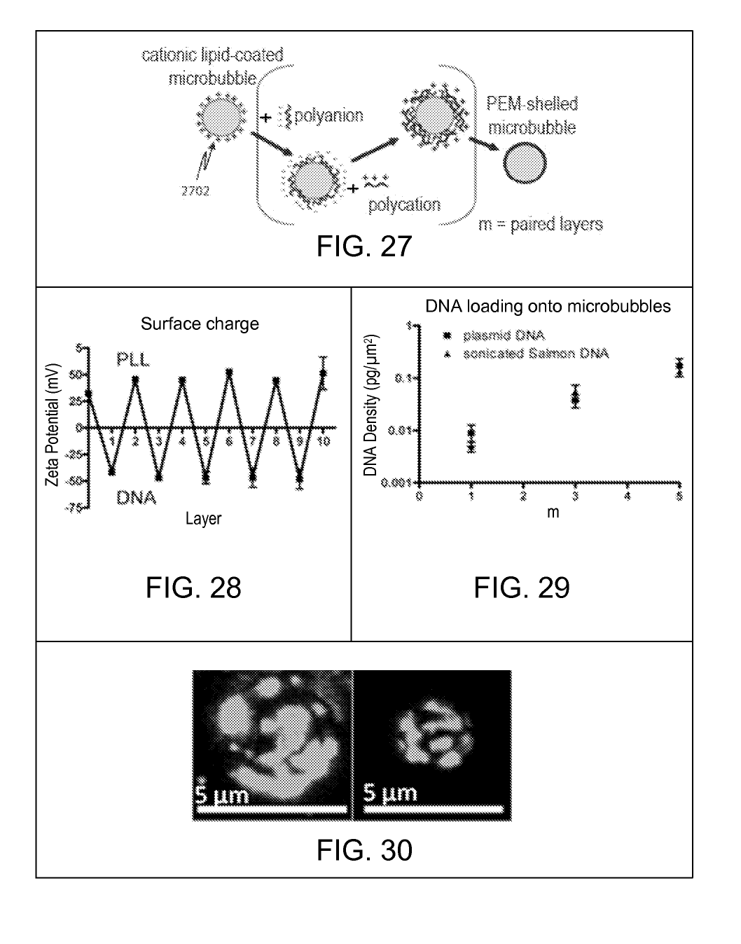

In embodiments, the loading capacity of microbubbles may be increased by using a layer-by-layer (LbL) assembly of a polyelectrolyte multilayer (PEM) composed of DNA and a biocompatible polycation to condense DNA and to increase the total available surface area of the microbubble. For example, the DNA loading capacity of a microbubble may be increased, by a factor of 10, by using an LbL assembly technique, as shown in FIG. 27. DNA, with its negatively charged phosphate groups, and polylysine, with its positively charged amine groups, can be sequentially adsorbed onto a cationic microbubble 2702 having a lipid shell containing, for example, DSTAP. The surface charge can oscillate stably between deposition steps, as shown in FIG. 28. Referring to FIG. 29, for five paired layers (e.g., 5 DNA+5 polylysine), the mass of DNA per unit area of microbubble surface can increase roughly tenfold over that of a single layer. In addition, multilayers can be formed as discrete domains on the microbubble surface, as shown in the images of FIG. 30. Oscillation and fragmentation may be possible during insonification at parameters used for imaging and/or drug delivery even with the presence of at least five paired layers.

In embodiments, the gas used to form these microbubbles can be perfluorobutane (PFB) at 99 wt % purity. DSPC and DSTAP can be dissolved in chloroform for storage. Other lipids may also be used as indicated herein. Polyoxyethylene-40 stearate (PEG40S) can be dissolved in deionized water. A fluorophore probe, such as 3,3′-dioctadecyloxacarbocyanine perchlorate (DiO) solution, can be used to label the microbubbles for microscopy and flow cytometry. The microbubbles shell can be formed from the DSPC, PEG-lipid (included ligand-bearing PEG-lipid) and DSTAP. The indicated amount of DSPC can be transferred to a glass vial, and the chloroform can be evaporated with a steady nitrogen stream during vortexing for about ten minutes followed by several hours under house vacuum. 0.01 M phosphate buffered saline (PBS) solution can be filtered using 0.2-μm pore size polycarbonate filters. The dried lipid film can then be hydrated with filtered PBS to a final lipid concentration of 1.0 mg/mL.

The lipid mixture can be sonicated with a 20-kHz probe at low power (e.g., approximately 3 W) in order to heat the pre-microbubble suspension above the main phase transition temperature of the phospholipid (e.g., approximately 55° C. for DSPC) and to further disperse the lipid aggregates into small, unilamellar liposomes. Sonication can be used for large microbubble batches, such as those used for size-isolation. PFB gas can be introduced by flowing it over the surface of the lipid suspension. Subsequently, higher power sonication (e.g., approximately 33 W) can be applied to the suspension for about 10 seconds at the gas-liquid interface to generate microbubbles. For flow cytometry and fluorescence microscopy experiments, DiO solution (1 mM) can be added prior to high-power sonication at an amount of 1 μL DiO solution per mL of lipid mixture.

Embodiments of the disclosed subject matter can result in an advanced gene delivery technology and can better characterize the underlying mechanisms of ultrasound-microbubble gene delivery. Moreover, systems, methods, and devices, as described herein may find particular benefit in the clinical treatment of pediatric cancer or other cancers. Although the description herein pertains generally to the delivery of plasmid DNA to targeted cells, the teachings of the present disclosure are applicable to other treatments as well. For example, the microbubbles described herein can be designed to carry synthetic oligonucleotides, siRNA, proteins, peptides, and/or other biological components. In addition, although particular configurations have been discussed herein, other configurations can also be employed.

Furthermore, the foregoing descriptions apply, in some cases, to examples generated in a laboratory, but these examples can be extended to production techniques. For example, where quantities and techniques apply to the laboratory examples, they should not be understood as limiting. In addition, although specific chemicals and materials have been disclosed herein, other chemicals and materials may also be employed according to one or more contemplated embodiments. For example, although the production of microbubbles with a hydrophobic gas has been specifically described herein, other gases (elemental or compositions) are also possible according to one or more contemplated embodiments

Features of the disclosed embodiments may be combined, rearranged, omitted, etc., within the scope of the invention to produce additional embodiments. Furthermore, certain features may sometimes be used to advantage without a corresponding use of other features.

It is thus apparent that there is provided in accordance with the present disclosure, system, methods, and devices for plasmid gene transfection using polymer-modified microbubbles. Many alternatives, modifications, and variations are enabled by the present disclosure. While specific embodiments have been shown and described in detail to illustrate the application of the principles of the present invention, it will be understood that the invention may be embodied otherwise without departing from such principles. Accordingly, Applicants intend to embrace all such alternatives, modifications, equivalents, and variations that are within the spirit and scope of the present invention.

1-23. (canceled)

24. A method for forming microbubbles for gene transfection comprising:
emulsifying a lipid formulation with a gas so as to produce a plurality of microbubble shells, each shell surrounding a respective gas-filled core region;
covalently attaching one or more polymers to each of the shells; and
electrostatically binding DNA to the one or more polymers so as to form one or more polyplex structures.

25. The method for forming microbubbles according to claim 24, wherein each respective core region is filled with a hydrophobic gas of SF₆ or perfluorobutane.

26. The method for forming microbubbles according to claim 24, wherein the lipid formulation comprises 90% DSPC, between 0.5% and 5% DSPE-PEG2K-Mal, and between 5% and 9.5% DSPE-PEG2K.

27. The method for forming microbubbles according to claim 24, wherein the one or more polymers comprise polyethyleneimine (PEI).

28. The method for forming microbubbles according to claim 27, further comprising, attaching one or more polyethylene glycol (PEG) polymer chains to the PEI.

29. The method for forming microbubbles according to claim 28, wherein the attaching one or more PEG polymer chains includes adding amine-reactive PEG succinimidyl ester at a 10:1 molar ratio to the PEI.

30. The method for forming microbubbles according to claim 24, further comprising, after the emulsifying, size-selecting the produced microbubbles such that the selected microbubbles have a diameter of 4-5 μm or 6-8 μm.

31. The method for forming microbubbles according to claim 24, wherein the covalently attaching includes:
thiolating the PEI to generate free sulfhydryl groups; and
covalently bonding the free sulfhydryl groups to maleimide of the microbubble shells.

32. The method for forming microbubbles according to claim 31, wherein said thiolating includes mixing 2-iminothiolane with the PEI at a 50:1 molar ratio.

33-34. (canceled)

35. A method of gene transfection, comprising:
injecting a plurality of microbubbles into a patient, each microbubble having a gas-filled core region surrounded by a shell, the shell being comprised of a lipid formulation and having one or more polyplex structures covalently attached thereto; and
applying a high-pressure, low-frequency ultrasound pulse to a region of interest in the patient so as to destroy microbubbles in said region of interest.

36. The method of claim 35, wherein the core region is filled with a hydrophobic gas of SF₆ or perfluorobutane, and the lipid formulation comprises 90% DSPC, between 0.5% and 5% DSPE-PEG2K-Mal, and between 5% and 9.5% DSPE-PEG2K.

37. The method of claim 35, wherein each polyplex structure includes polyethyleneimine (PEI) with DNA electrostatically bound thereto.

38. The method of claim 37, wherein each polyplex structure includes a polyethylene glycol (PEG) polymer chain attached to the PEI.

39. The method of claim 35, wherein the plurality of microbubbles have diameters of 4-5 μm or 6-8 μm.

40. The method of claim 35, wherein sulfhydryl of each polyplex structure is covalently attached to maleimide of the respective microbubble shell.

41. The method of claim 35, wherein said applying is such that DNA carried by the destroyed microbubbles is introduced into cells in the region of interest.

42. The method of claim 41, wherein the DNA is introduced into the cells by sonoporation or by endocytotic uptake of the polyplex structures.

43. (canceled)

44. The method of claim 35, further comprising imaging vasculature in the region of interest using ultrasound.

45. The method of claim 44, wherein said imaging includes using the microbubbles in the region of interest as an ultrasound contrast agent.

46. The method of claim 35, wherein said region of interest includes a cancerous tumor.
